(19) United States
(12) Patent Application Publication
(10) Pub. No.: US 2024/0149071 A1
(43) Pub. Date: May 9, 2024

(54) MAGNET DEVICE FOR RELIEVING ACHES AND/OR PAIN

(71) Applicant: Diane A. Soracco, Flint, MI (US)

(72) Inventor: Diane A. Soracco, Flint, MI (US)

(21) Appl. No.: 18/213,807

(22) Filed: Jun. 23, 2023

Related U.S. Application Data
(60) Provisional application No. 63/355,092, filed on Jun. 23, 2022.

(51) Int. Cl. A61N 2/00; A61N 2/06; H01F 7/02

(52) U.S. Cl. CPC A61N 2/008; A61N 2/06; H01F 7/02

(57) ABSTRACT

The present invention is a magnet device for relieving aches and/or pain. One non-limiting embodiment is a magnet device for relieving aches and/or pain of a particular body area of a user wherein magnet device includes a plurality of magnets arranged end to end in close proximity to one another in a generally linear configuration, and a wire, a rod or an elongated extension for securing the magnets together in a generally linear configuration. Another non-limiting embodiment is a magnet device for relieving aches and/or pain of a particular body area of a user wherein magnet device includes a plurality of magnets arranged end to end in close proximity to one another, and a body brace or pad adapted for securing at least one linear configuration of the magnets to or inside the body brace or pad. This embodiment may also include a wire, a rod or an elongated extension.

### CROSS-REFERENCE TO RELATED APPLICATIONS

The present application claims the priority benefit of U.S. Provisional Patent Application Ser. No. 63/355,092, filed on Jun. 23, 2022 and titled “MAGNET DEVICE FOR RELIEVING ACHES AND/OR PAIN,” which is incorporated herein by reference in its entirety.

### BACKGROUND OF THE INVENTION

### Field of the Invention

The present invention generally relates to magnet devices for relieving aches and/or pain. More particularly, the present invention is a magnet device comprising a plurality of magnets positioned end to end in close proximity to one another (preferably abutting or touching one another) in a generally linear configuration (or configuration line) for relieving aches and/or pain of a particular body area of a user. In use, the magnet device is positioned or placed on, beside, next to or in close proximity to a particular body area of a user that desires to receive relief of aches and/or pain.

### Description of the Related Art

Magnet devices for relieving aches and/or pain are known in the art.

The present invention is a magnet device for relieving aches and/or pain of a particular body area of a user.

The present invention overcomes one or more of the shortcomings of magnet devices for relieving aches and/or pain. The Applicant is unaware of inventions or patent documents, taken either singly or in combination, which are seen to describe the present invention as claimed.

One advantage of the present invention is a non-medicinal or non-chemical method to relieve aches and/or pain of a particular body area of a user.

Another advantage of the present invention is a cost effective device to relieve aches and/or pain of a particular body area of a user.

### SUMMARY OF THE INVENTION

One non-limiting embodiment of the present invention is a magnet device for relieving aches and/or pain of a particular body area of a user wherein the magnet device includes a plurality of magnets arranged end to end in close proximity to one another (preferably abutting or touching one another) in a generally linear configuration (or configuration line), and a wire, a rod or an elongated extension for securing the magnets together in a generally linear configuration (or configuration line).

Another non-limiting embodiment of the present invention is a magnet device for relieving aches and/or pain of a particular body area of a user wherein the magnet device includes a plurality of magnets arranged end to end in close proximity to one another (preferably abutting or touching one another) in a generally linear configuration (or configuration line), and a body brace or pad adapted for securing at least one linear configuration of the magnets to or inside the body brace or pad. This embodiment may also include a wire, a rod or an elongated extension.

In use, the magnet device is positioned or placed on, beside, next to or in close proximity to a particular body area of a user that desires to receive relief of aches and/or pain.

### BRIEF DESCRIPTION OF THE DRAWINGS

FIG. 1 is a perspective view of an embodiment of a magnet device for relieving aches and/or pain of a particular body area of a user according to the present invention;

FIG. 2 is a perspective view of another embodiment of a magnet device for relieving aches and/or pain of a particular body area of a user according to the present invention;

FIG. 3 is a perspective view of an additional embodiment of a magnet device for relieving aches and/or pain of a particular body area of a user according to the present invention;

FIG. 4 is a perspective view of a further embodiment of a magnet device for relieving aches and/or pain of a particular body area of a user according to the present invention;

FIG. 5 is a perspective view of another embodiment of a magnet device for relieving aches and/or pain of a particular body area of a user according to the present invention;

FIG. 6 shows regular magnet poles;

FIG. 7 shows a wave produced by same side polls resisting; and

FIGS. 8-13 show how the waves produced by opposing poles (for S-N-N-S-S-N-N . . . as compared for S-N-S-N-S-N-S . . . ) to penetrate through the body and relieve aches and/or pain.

It should be understood that the above-attached figures are not intended to limit the scope of the present invention in any way.

### DETAILED DESCRIPTION OF THE EMBODIMENTS OF THE PRESENT INVENTION

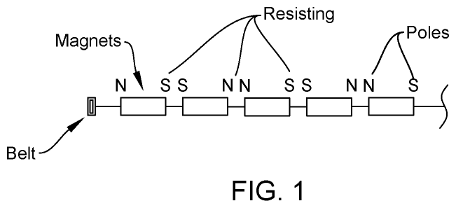

Referring to FIGS. 1-3, the present invention is a magnet device for relieving aches and/or pain of a particular body area of a user. One non-limiting embodiment of the present invention is a magnet device 100,200,300 (such as, but not limited to, a bracelet 200, a necklace, an anklet, or a vertical pile 300) that includes a plurality of magnets 110′,110″,110″′,110(END),210′,210″,210″′, 210(END),310′,310″,310″′, 310(END) arranged end to end in close proximity to one another (preferably abutting or touching one another), and a wire, a rod or an elongated extension 150,250 (preferably not attracted to magnets 110′,110″, 110″′,110(END),210′, 210″,210″′,210(END),310′,310″,310″′,310(END)) for securing the multiple magnets 110′,110″,110″′,110(END),210′,210″,210″′, 210(END),310′,310″,310″′, 310(END) together in a generally linear configuration (or configuration line). Preferably, the magnets 110′,110″,110″′,110(END),210′, 210″,210″′,210(END), 310′,310″,310″′,310(END) are positioned end to end in close proximity to one another (preferably abutting or touching one another) wherein the first end 112′,212′,312′ (“north pole” end) of a first magnet 110′, 210′,310′ (magnet #1) is adjacent to the first end 112″,212″,312″ (“north pole” end) of an adjacent end-to-end magnet 110″,210″,310″ (magnet #2) while the second end 114′,214′,314′ (“south pole” end) of the first magnet 110′,210′,310′ is adjacent to the second end 114″′,214″′,314″′ (“south pole” end) of a different adjacent end-to-end magnet 110″′,210″′,310″′ (magnet #3), and so on.

Non-limiting examples of a wire, a rod or an elongated extension 150, 250,350 are an aluminum wire, an aluminum bolt, or an aluminum bolt and nut device. The wire, rod or elongated extension 150,250.350 may also be made or manufactured of metal, steel, rubber, plastic, a cloth material, and any combination thereof. Preferably, the wire, rod or elongated extension 150,250, 350 may be made or manufactured of a materials) that is not attracted to the magnets 110′,110″,110″′,110(END),210′,210″,210″′,210(END),310′,310″,310″′, 310(END). As a non-limiting example, the wire, rod or elongated extension 150,250,350 may be made or manufactured of a material(s) that is not attracted to the magnets 110′,110″,110″′,110(END),210′, 210″,210″′,210(END),310′,310″, 310″′,310(END) due to its crystal structure.

FIG. 1 shows a magnet device 100 that includes a plurality of magnets 110′,110″,110″′,110(END) arranged end to end in close proximity to one another, and a bolt 150 for securing the magnets 110′,110″,110″′,110(END) together in a generally linear configuration (or configuration line). In this non-limiting example, the magnets 110′,110″,110″′,110(END) (such as, but not limited to, neodyme 52 magnets) are being ready to be positioned or pressed together to form a pressed, resisting, alternating pole connected energy line that is able to decrease pain, swelling, and/or bacterial growth by using the resistance force of the magnets 110′,110″,110″′,110(END) to positively effect the applicable body area(s) of the user.

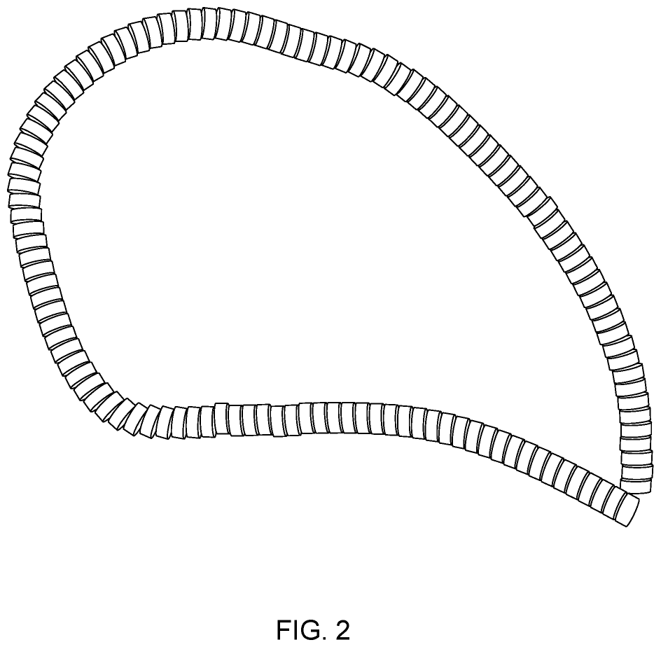

FIG. 2 shows a magnet device in the configuration of a bracelet 200. The device 200 includes a plurality of magnets 210′,210″,210″′,210(END) arranged end to end and abutting or touching one another, and a wire, a rod or an elongated extension 250 for securing the magnets 210′,210″,210″′,210(END) together in a generally linear configuration (or configuration line) for a certain distance along the bracelet 200.

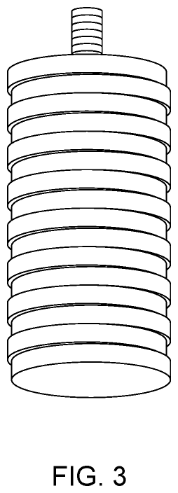

FIG. 3 shows a magnet device in the configuration of a vertical pile 300. The device 300 includes a plurality of magnets 310′,310″,310″′,310(END) arranged end to end and abutting or touching one another, and a bolt 350 for securing the magnets 310′,310″,310″′,310(END) together in a generally linear configuration (or configuration line).

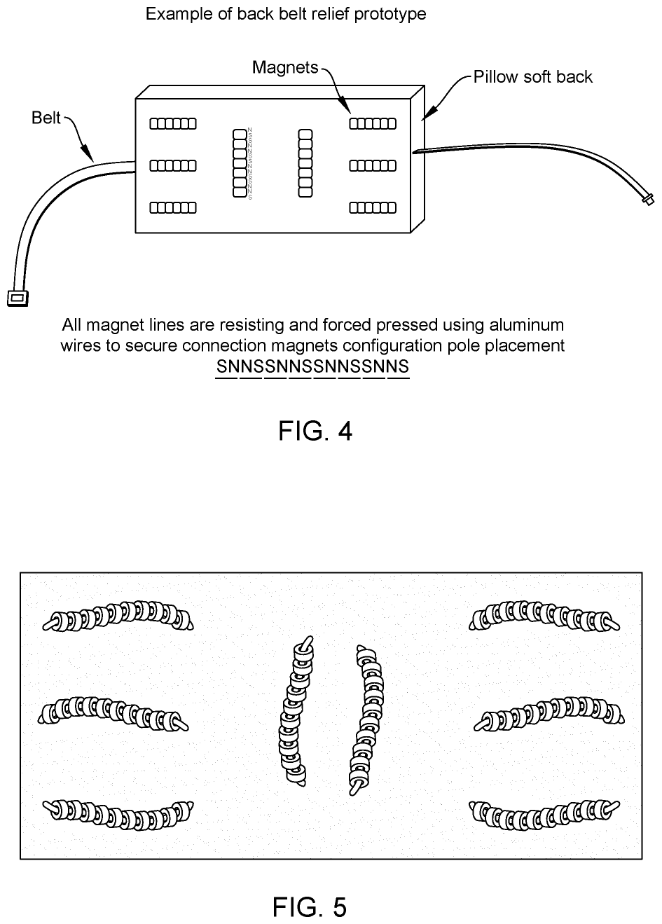

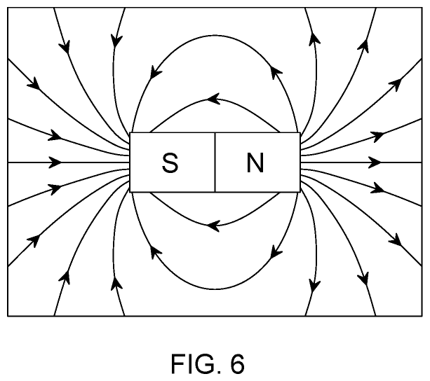

Referring to FIGS. 4-5, another non-limiting embodiment of the present invention is a magnet device 400 (such as, but not limited to, a back brace or back belt relief device 400, an arm brace, a knee brace, an ankle brace or a body pad) that includes a plurality of magnets 410′,410″,410″′,410(END) arranged end to end in close proximity to one another (preferably abutting or touching one another), and a body brace or pad 470 (with a belt 490) adapted for securing at least one linear configuration (preferably multiple linear configurations) of the magnets 410′,410″,410″′, 410(END) to or inside the body brace or pad 470. This embodiment may also include a wire, a rod or an elongated extension 450.

Preferably, the magnets 410′,410″,410″′,410(END) are positioned end to end in close proximity to one another (preferably abutting or touching one another) wherein the first end 412′,412′,412′ (“north pole” end) of a first magnet 410′ (magnet #1) is adjacent to the first end 412″ (“north pole” end) of an adjacent end-to-end magnet 410″ (magnet #2) while the second end 114′ (“south pole” end) of the first magnet 110′ is adjacent to the second end 114″′ (“south pole” end) of a different adjacent end-to-end magnet 110″′ (magnet #3), and so on.

The body brace or pad 470 may include slots, pockets, cavities, tape, glue, adhesive, or other securing elements (such as, but not limited to, a hook and loop device) for securing the linear configuration(s) of magnets 410′,410″, 410″′,410(END) to or inside the body brace or pad 470.

Non-limiting examples of a wire, a rod or an elongated extension 450 are an aluminum wire, an aluminum bolt, or an aluminum bolt and nut device. The wire, rod or elongated extension 450 may also be made or manufactured of metal, steel, rubber, plastic, a cloth material, and any combination thereof. Preferably, the wire, rod or elongated extension 450 may be made or manufactured of a materials) that is not attracted to the magnets 410′,410″,410″′, 410(END). As a non-limiting example, the wire, rod or elongated extension 450 may be made or manufactured of a material(s) that is not attracted to the magnets 410′,410″,410″′,410(END) due to its crystal structure.

As shown in FIG. 4, the body brace or pad 470 may be a pillow soft back pad, and multiple linear configurations of magnets 410′,410″,410″′,410(END) are secured to the back pad 470 in certain or predetermined locations where the pressed, resisting, alternating pole connected energy lines is able to decrease pain, swelling, and/or bacterial growth by using the resistance force of the magnets 410′,410″,410″′,410(END) to positively effect the applicable body area(s) of the user.

In use, the magnet device 100,200,300,400 is positioned or placed on, beside, next to or in close proximity to a body area of a user that is desired to have relief of aches and/or pain.

FIG. 6 shows regular magnet poles.

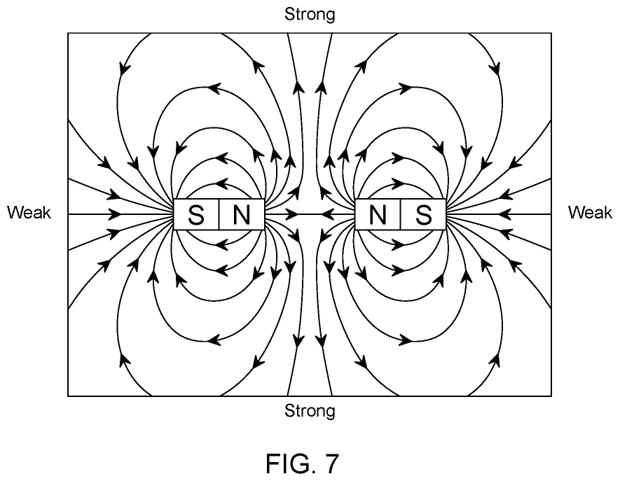

FIG. 7 shows a wave produced by same side polls resisting, as with the present invention.

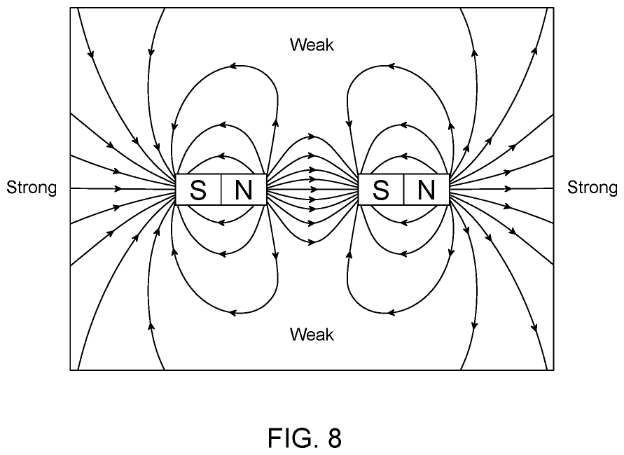

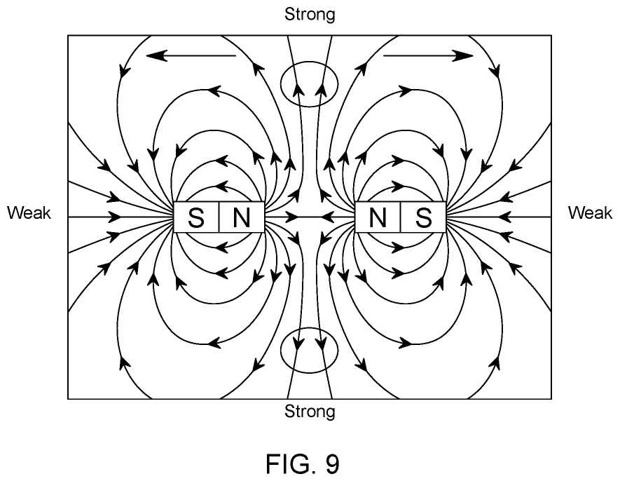

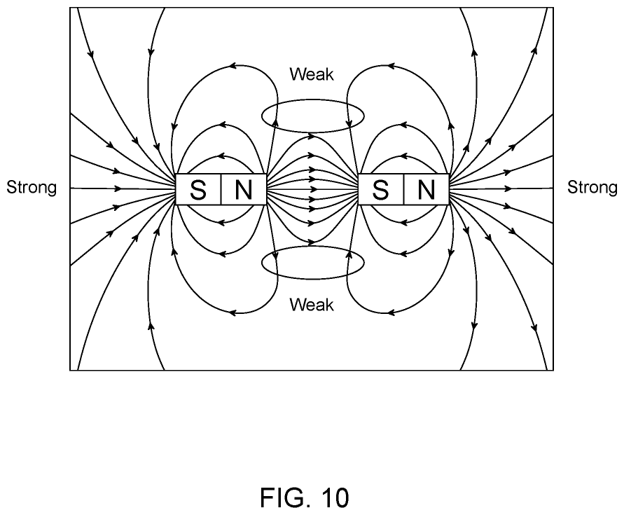

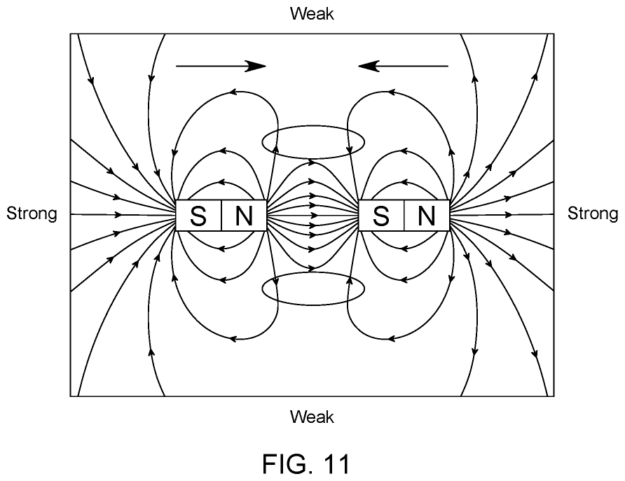

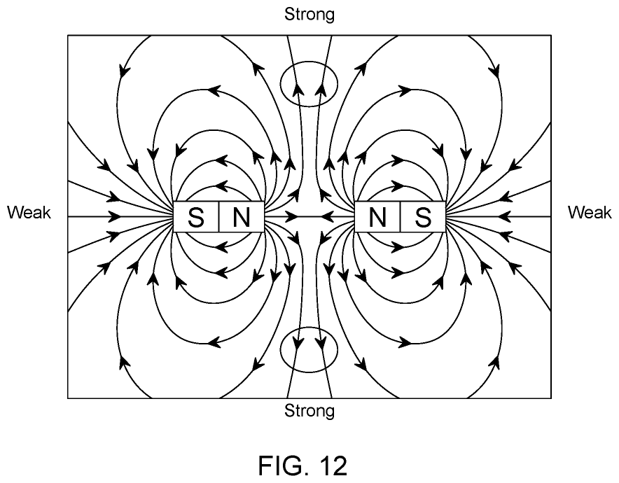

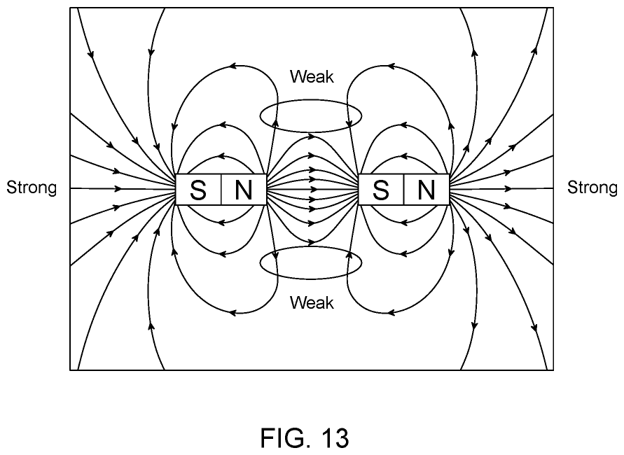

FIGS. 8-13 show how the waves produced by opposing poles (for S-N-N-S-S-N-N . . . as compared for S-N-S-N-S-N-S . . . ) to penetrate through the body and relieve aches and/or pain.

It is to be understood that the present invention is not limited to the embodiments described above or as shown in the attached figures, but encompasses any and all embodiments within the spirit of the invention.

1. A magnet device for relieving aches and/or pain of a particular area of a body, said magnet device comprising:
a plurality of magnets arranged end to end in close proximity to one another in a generally linear configuration; and
an elongated extension for securing said plurality of magnets together in a generally linear configuration.

2. The magnet device according to claim 1, wherein each magnet of said plurality of magnets abut or touching at least one adjacent magnet of said plurality of magnets.
